(19) United States
(12) Patent Application Publication
(10) Pub. No.: US 2024/0424256 A1
(43) Pub. Date: Dec. 26, 2024

(54) CATHETERIZATION ROBOT

(71) Applicant: ROBOCATH, ROUEN (FR)

(72) Inventor: Matthieu GILLES, ROUEN (FR)

(21) Appl. No.: 18/692,856

(22) PCT Filed: Sep. 15, 2022
(86) PCT No.: PCT/EP2022/075659

§ 371 Date: Mar. 18, 2024

(30) Foreign Application Priority Data
Sep. 17, 2021 (FR) FR2109766

(51) Int. Cl. A61M 25/01; A61M 39/10

(52) U.S. Cl. CPC A61M 25/0113; A61M 39/105

(57) ABSTRACT

The invention relates to a catherization robot (1) comprising: a base (10), a first drive module (2) for driving a first elongate flexible medical instrument (4) in longitudinal translation, said first drive module being fixed relative to the base (10) and comprising at least one pair of two drive surfaces (20) which are located face to face and which: either move closer to each other another in transverse translation along a first direction, firstly so as to grip the first elongate flexible medical instrument (4) in order to then be able to drive it in longitudinal translation, or move away from each other in transverse translation along a second direction opposite to the first direction, in order to release the first elongate flexible medical instrument (4) so it is then free for extraction from the catherization robot (1), the maximum value of the separation distance between the two drive surfaces (20) of the first drive module (2), which is reached when the two drive surfaces (20) move apart from each other, being greater than 20 mm.

### FIELD OF THE INVENTION

The invention relates to the field of catherization robots, and in particular the field of catherization robots comprising at least a first drive module that is fixed relative to the base of the catherization robot and a second drive module that is movable in longitudinal translation relative to the fixed first drive module.

### TECHNOLOGICAL BACKGROUND OF THE INVENTION

From the prior art according to the patent application filed under number FR2101183 on Feb. 8, 2021, a catherization robot is known comprising at least a first drive module that is fixed relative to the base of the catherization robot and a second drive module mobile that is movable in longitudinal translation relative to the first drive module.

Such a catherization robot drives, in longitudinal translation, an elongate flexible medical instrument which comprises a body intended to enter the body of a patient and a manipulation area which is not intended to enter the body of the patient and which allows gripping the elongate flexible medical instrument.

The manipulation area of the elongate flexible medical instrument is wider than the body of the elongate flexible medical instrument. The body of the elongate flexible medical instrument passes longitudinally through the first drive module, while the manipulation area of the elongate flexible medical instrument, which is wider, generally much wider, is stopped in front of the first drive module.

The invention highlights a disadvantage of this prior art:
the length of the body of the elongate flexible medical instrument which remains inside the first drive module, when the manipulation area of the elongate flexible medical instrument is stopped in front of the first drive module, is a useless length that is wasted,
the greater the length of the body of the elongate flexible medical instrument which remains inside the first drive module when the manipulation area of the elongate flexible medical instrument is stopped in front of the first drive module, the greater the useless and wasted length of the body of the elongate flexible medical instrument,
the useless and wasted length will be even greater if, behind the manipulation area of the elongate flexible medical instrument, a Y connector is added within which the body of the elongate flexible medical instrument also moves in longitudinal translation.

### OBJECTS OF THE INVENTION

The aim of the present invention is to provide a catherization robot which at least partially overcomes the above disadvantages.

More particularly, the invention aims to provide a catherization robot which will make better use of the body length of the elongate flexible medical instrument which was useless and wasted in the prior art, by simply extending the maximum traveled distance between drive surfaces of the first drive module.

The invention will then obtain a net gain of a substantial portion of the body length of the elongate flexible medical instrument, with a simple solution.

To this end, the invention proposes a catherization robot comprising: a base; a first drive module for driving a first elongate flexible medical instrument in translation along a longitudinal first direction, said first drive module being fixed relative to the base and comprising at least one pair of two drive surfaces which are located face to face and which: either move closer to each other in translation along a transverse second direction, in a first directional orientation, firstly so as to grip the first elongate flexible medical instrument in order to then be able to drive it in translation along the longitudinal first direction, or move away from each other in translation along the transverse second direction, in a second directional orientation that is opposite to the first directional orientation, in order to release the first elongate flexible medical instrument so it is then free for extraction from the catherization robot; a second drive module for driving a second elongate flexible medical instrument in translation along the longitudinal first direction, said second drive module being movable in translation along the longitudinal first direction relative to the first drive module, one end of the first elongate flexible medical instrument being fixed to the second drive module, characterized in that: the maximum value of the separation distance between the two drive surfaces of the first drive module, which is reached when the two drive surfaces are moving away from each other, is greater than 20 mm, or greater than 25 mm, or greater than 30 mm, or greater than 35 mm.

To this end, the invention also proposes a catherization robot comprising: a base; a first elongate flexible medical instrument; a first drive module for driving in translation, along a longitudinal first direction, a first elongate flexible medical instrument comprising a body which is intended to enter the body of a patient and a manipulation area which is intended to remain outside the body of the patient and which allows gripping this first elongate flexible medical instrument, said manipulation area having a maximum transverse dimension that is greater than the maximum transverse dimension of the body of the first elongate flexible medical instrument, the respective transverse dimensions of the manipulation area and of the body of the first medical instrument being oriented in a transverse second direction which is orthogonal to the longitudinal first direction, said first drive module being fixed relative to the base, and comprising at least a pair of two drive surfaces which are located face to face and which: either move closer to each other in translation along the transverse second direction, in a first directional orientation, firstly so as to grip the first elongate flexible medical instrument in order to then be able to drive it in translation along the longitudinal first direction, or move away from each other in translation along the transverse second direction, in a second directional orientation that is opposite to the first directional orientation, in order to release the first elongate flexible medical instrument so it is then free for extraction from the catherization robot, the separation distance between the two drive surfaces then reaching a maximum value which is greater than said transverse dimension of said body of the first elongate flexible medical instrument; a second drive module for driving a second elongate flexible medical instrument in translation along the longitudinal first direction, said second drive module being movable in translation along the longitudinal first direction relative to the first drive module, one end of the first elongate flexible medical instrument being fixed to the second drive module,

characterized in that: said maximum value of the separation distance between the two drive surfaces of the first drive module is greater than said maximum transverse dimension of said manipulation area of the first elongate flexible medical instrument.

To this end, the invention also proposes a catherization robot comprising: a base; a Y connector; a first drive module for driving a first elongate flexible medical instrument in translation along a longitudinal first direction, said first drive module being fixed relative to the base and comprising at least one pair of two drive surfaces which are located face to face and which: either move closer to each other in translation along the transverse second direction, in a first directional orientation, firstly so as to grip the first elongate flexible medical instrument in order to then be able to drive it in translation along the longitudinal first direction, or move away from each other in translation along the transverse second direction, in a second directional orientation that is opposite to the first directional orientation, in order to release the first elongate flexible medical instrument so it is then free for extraction from the catherization robot; a second drive module for driving a second elongate flexible medical instrument in translation along the longitudinal first direction, said second drive module being movable in translation along the longitudinal first direction relative to the first drive module, one end of the first elongate flexible medical instrument being fixed to the second drive module, and said second drive module carrying the Y connector within which the second elongate flexible medical instrument can move in translation along the longitudinal first direction,

characterized in that: the maximum value of the separation distance between the two drive surfaces of the first drive module, reached when the two drive surfaces are moving away from each other, is greater than the maximum transverse dimension of the Y connector along the transverse second direction.

To this end, the invention also proposes a method for moving an elongate flexible medical instrument in a catherization robot, successively comprising: a first step in which the body of the first elongate flexible medical instrument is driven in translation along the longitudinal first direction by at least one pair of drive surfaces, and a second step in which the body of the first elongate flexible medical instrument is driven in translation along the longitudinal first direction by a translational movement of the second drive module relative to the first drive module along the longitudinal first direction.

Preferably, the first step comprises: a first sub-step in which the body of the first elongate flexible medical instrument is driven in translation along the longitudinal first direction by at least two of said pairs of drive surfaces, and a second sub-step in which the body of the elongate flexible medical instrument is driven in translation along the longitudinal first direction by only one of said pairs of drive surfaces.

This method for moving an elongate flexible medical instrument in a catherization robot can be used for example to train doctors in the operation of the catherization robot, by inserting the elongate flexible medical instrument into a mannequin which is an object simulating a patient.

To this end, the invention also proposes a use, in a method for therapeutic treatment or in a method for diagnosing a patient, of the method for moving an elongate flexible medical instrument in a catherization robot according to the invention and/or of the catherization robot according to the invention.

According to preferred embodiments, the invention comprises one or more of the following features which may be used separately or in combinations of some of them or in a combination of all of them, with any of the previously cited objects of the invention.

Preferably, said maximum value of the separation distance between the two drive surfaces is: greater than 1.25 times said maximum transverse dimension of said manipulation area of the first elongate flexible medical instrument, or greater than 1.5 times said maximum transverse dimension of said manipulation area of the first elongate flexible medical instrument, or greater than 1.25 times said maximum transverse dimension of the Y connector, or greater than 1.5 times said maximum transverse dimension of the Y connector.

There is thus even less risk of the elongate flexible medical instrument catching on part of the drive mechanism of the first drive module.

Preferably, said maximum value of the separation distance between the two drive surfaces is: less than 3 times said maximum transverse dimension of said manipulation area of the first elongate flexible medical instrument, or less than 2 times said maximum transverse dimension of said manipulation area of the first elongate flexible medical instrument, or less than 3 times said maximum transverse dimension of the Y connector, or less than 2 times said maximum transverse dimension of the Y connector, or less than 80 mm, or less than 60 mm, or less at 1050 mm, or less than 40 mm.

The very slight additional cost that may be incurred by extending the maximum traveled distance between drive surfaces of the first drive module is thus further reduced.

Preferably, the catherization robot comprises: an inlet opening in the body of the first elongate flexible medical instrument, its transverse dimension in the transverse second direction being greater than said maximum transverse dimension of said manipulation area of the first elongate flexible medical instrument, or greater than said maximum transverse dimension of the Y connector if there is such; an outlet opening in the body of the first elongate flexible medical instrument, its transverse dimension in the transverse second direction being greater than said maximum transverse dimension of said manipulation body of the first elongate flexible medical instrument, or greater than said maximum transverse dimension of said manipulation area of the first elongate flexible medical instrument, or less than said maximum transverse dimension of the Y connector if there is such.

Preferably, said manipulation area of the first elongate flexible medical instrument comprises one or more protuberances arranged in a manner that enables gripping the first elongate flexible medical instrument, said protuberances advantageously being fins.

Preferably, said drive surfaces are part of a consumable interface of the first drive module, said consumable interface advantageously having the shape of a plate or of a flat closed casing.

Preferably, said two drive surfaces are respectively carried by two rollers which rotate about two parallel axes but in directions opposite to each other and which are part of the first drive module.

Preferably, said two drive surfaces are respectively carried by two endplates which are movable in translation.

Preferably, said two drive surfaces are respectively carried by two platforms which are movable in transverse translation along the transverse second direction, and which are part of the first drive module which also comprises an actuator for driving said two platforms in translation along the transverse second direction.

Preferably, the actuator is formed by a worm screw system.

Preferably, the actuator is formed by an actuating cylinder.

Preferably, the first drive module comprises at least two of said pairs of two drive surfaces, which are located one after the other along the longitudinal first direction.

There is thus even less wasted body length of the elongate flexible medical instrument.

Preferably, the first elongate flexible medical instrument is a catheter or a guide catheter, and the second elongate flexible medical instrument is a catheter guide or a catheter.

Other features and advantages of the invention will become apparent upon reading the following description of a preferred embodiment of the invention, given by way of example and with reference to the appended drawings.

### BRIEF DESCRIPTION OF DRAWINGS

FIG. 1 schematically represents an example of a catherization robot according to one embodiment of the invention.

FIG. 2 schematically represents an example of a first step in moving a medical instrument according to one embodiment of the invention.

FIG. 3 schematically represents an example of a second step in moving a medical instrument according to one embodiment of the invention.

FIG. 4 schematically represents an example of a third step in moving a medical instrument according to one embodiment of the invention.

### DETAILED DESCRIPTION OF EMBODIMENTS OF THE INVENTION

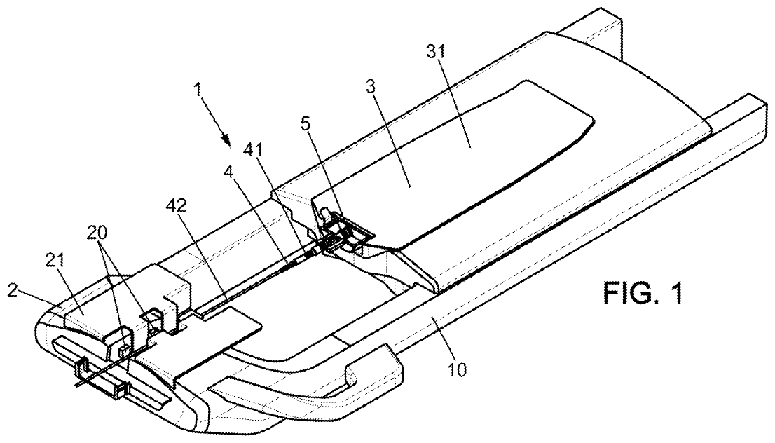

As illustrated in FIG. 1, a catherization robot 1 comprises a base 10 on which are mounted a fixed drive module 2 and a drive module 3 which is movable in translation relative to fixed drive module 2. Fixed drive module 2 and movable drive module 3 each comprise means for driving an elongate flexible medical instrument in translation and in rotation. These means for driving the medical instrument are formed by at least one pair of drive surfaces which are positioned face to face. These drive surfaces may for example be formed by endplates carried by robotic fingers, as described in patent FR3022147, or the patent application filed under number FR2014222 on Dec. 29, 2020. These drive surfaces may also be formed by rotating rollers.

Fixed drive module 2 comprises a consumable interface 21 in the form of a plate or cassette which is intended to receive a guide catheter or a micro-catheter, or a catheter which carries a balloon or stent. Movable drive module 3 also comprises a consumable interface 31 in the form of a plate or cassette which is intended to receive a catheter guide and a catheter which carries a balloon or stent. Consumable interfaces 21 and 31 may in particular comprise consumable endplates 20 which are intended to form a sterile interface between the fingers of fixed and movable modules 2 and 3 and the elongate flexible medical instruments, as described in patent FR3022147. For simplification purposes, endplates 20 are only shown for fixed drive module 2.

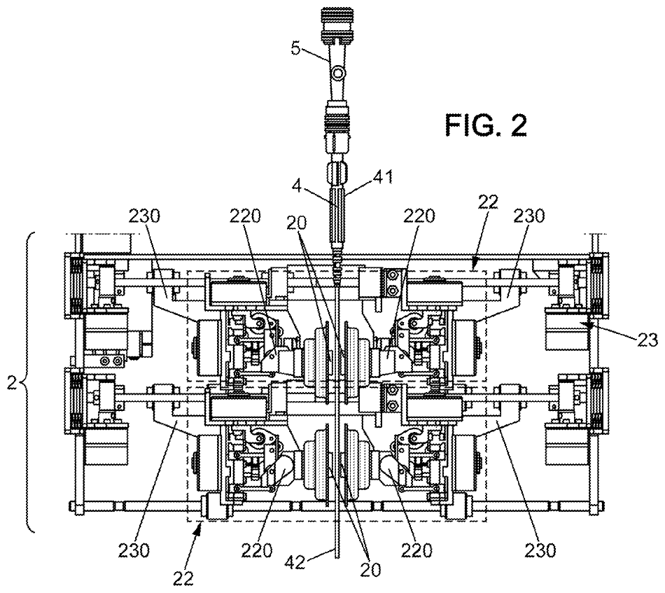

An elongate flexible medical instrument 4, which may be a guide catheter or a micro-catheter or a catheter, is installed on consumable interface 21 in order to be manipulated by fixed drive module 2. To do so, the proximal end of medical instrument 4 is fixed to a Y connector 5 which is installed on the distal end of consumable interface 31 of movable drive module 3. As can be seen in FIGS. 2 to 4, a manipulation area 41 is located on the proximal end of medical instrument 4, this manipulation area 41 being formed by an increase in the diameter of medical instrument 4 and possibly comprising fins. Manipulation area 41 is also called “hub”. Medical instrument 4 also comprises a body 42 which is intended to be inserted into the patient's body, unlike manipulation area 41 which is intended to remain outside the patient. As the diameter of manipulation area 41 is greater than the diameter of body 42, manipulation area 41 having a maximum transverse dimension greater than the maximum transverse dimension of body 42, the respective transverse dimensions of manipulation area 41 and of body 42 are oriented in a transverse second direction which is orthogonal to a longitudinal first direction. The longitudinal first direction corresponds to the main direction of elongation of medical instrument 4.

FIGS. 2 to 4 show different steps in the movement of medical instrument 4 within fixed drive module 2, the consumable interface 21 not being shown in order to facilitate understanding of the operation of fixed drive module 2.

FIG. 2 shows the situation illustrated in FIG. 1, where manipulation area 41 is located outside fixed drive module 2, only body 42 being positioned in fixed drive module 2. In the situation illustrated in FIG. 2, fixed drive module 2 is manipulating medical instrument 4 by means of body 42.

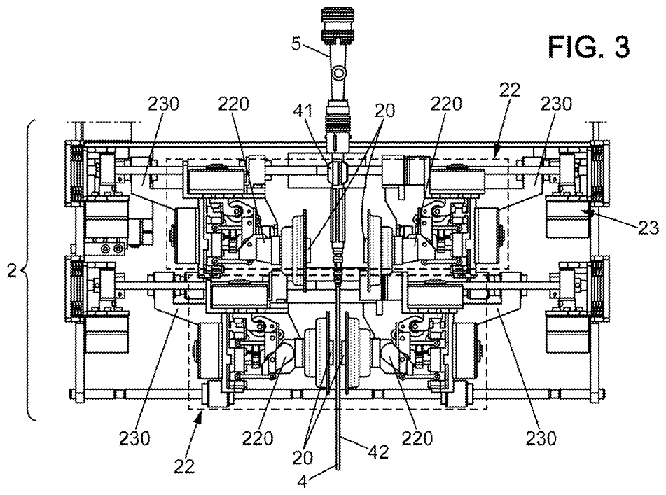

FIG. 3 shows the situation where medical instrument 4 is inserted far enough into the patient's body for manipulation area 41 to reach the proximal end of fixed drive module 2 and to enter fixed drive module 2.

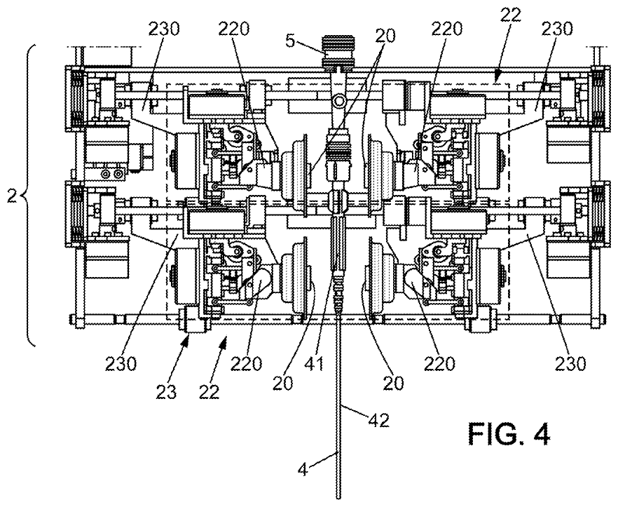

FIG. 4 shows the situation where medical instrument 4 is inserted far enough into the patient's body for manipulation area 41 to be inserted all the way inside fixed drive module 2 and Y connector 5 has also entered into fixed drive module 2.

In the variant illustrated in the figures, fixed module 2 comprises two manipulation devices 22 which are configured to drive medical instrument 4 in translation along its main axis of elongation, and to drive medical instrument 4 in rotation about its main axis of elongation. Each manipulation device 22 is formed by a pair of manipulator fingers 220, as described in the patent application filed under number FR2014222 on Dec. 29, 2020.

In order to manipulate medical instrument 4, manipulator fingers 220 of each pair must be brought together in order to clasp body 42 of medical instrument 4. This arrangement corresponds to what is illustrated in FIG. 2. In this first step in the operation of catherization robot 1, endplates 20 of manipulator fingers 220 of each pair can move apart by a distance of between 7 mm and 3 mm, preferably between 6 mm and 4 mm, and preferably 5 mm. This distance corresponds to the moment when the pair of manipulator fingers 220 stops clasping medical instrument 4 to return to their position, as described for example in the patent application filed under number PCT/IB2020/001134 on Dec. 26, 2020.

When medical instrument 4 is inserted far enough that manipulation area 41 reaches the proximal pair of manipulator fingers 220, a spacing device 23 for moving apart manipulator fingers 220 is actuated in order to move manipulator fingers 220 apart to allow the passage of manipulation area 41. When the proximal pair of manipulator fingers 220 is moved apart it stops manipulating medical instrument 4. Thus, in FIG. 3, only the distal pair of manipulator fingers 220 is manipulating medical instrument 4. The separation distance between endplates 20 of manipulator fingers 220 is sufficiently great to allow the passage of manipulation area 41 and Y connector 5. To do so, in this second step in the operation of catherization robot 1, endplates 20 of manipulator fingers 220 of the proximal pair can move apart by at least 20 mm, preferably at least 25 mm, preferably 30 mm, and preferably at least 35 mm. In this second step of operation, manipulator fingers 220 of the distal pair continue to be moved only slightly apart during the return travel, as described for the first step in the operation of catherization robot 1.

As can be seen in FIG. 4 illustrating the third step in the operation of catherization robot 1, the distal pair of manipulator fingers 220 can also be moved apart by the spacing device 23 in order to allow the passage of manipulation area 41 of medical instrument 4 when said medical instrument 4 is inserted far enough into the patient's body that the manipulation area reaches the distal pair of manipulator fingers 220. When the distal pair of manipulator fingers 220 is moved away, it stops manipulating medical instrument 4. Thus, in FIG. 4, medical instrument 4 can only be pushed or pulled, causing translational movement of movable module 3 in order to push or pull Y connector 5 to which medical instrument 4 is attached.

When medical instrument 4 is withdrawn from the patient's body, whether partially or completely, spacing device 23 will be reactivated in order to tighten the different pairs of manipulator fingers 220 once the pair of manipulator fingers 220 is located facing body 42 of medical instrument 4 and is no longer facing manipulation area 41 or Y connector 5. Once tightened, the pair of manipulator fingers 220 can once again grip and manipulate medical instrument 4.

Spacing device 23 comprises a platform 230 that is movable in transverse translation along the transverse second direction for each manipulator finger 220. In order to move these platforms 230, spacing device 23 comprises a worm screw system: for each of the platforms 230, a threaded rod is rotated by a motor in order to move translationally platform 230 linked to the rod. However, the worm screw system is only one possible variant for moving the pair of manipulator fingers 220 apart or closer together; a solution with actuator cylinders is also possible.

The solution proposed by the invention is not limited to the manipulation of medical instrument 4 with manipulator fingers 220. The invention may for example be applied to the manipulation of medical instrument 4 by rotating rollers, where the rollers can be moved apart in order to allow the passage of manipulation area 41 of medical instrument 4 and the Y connector 5.

Of course, the invention is not limited to the examples and to the embodiment described and represented, but is capable of numerous variants accessible to those skilled in the art.

1-15. (canceled)

16. Catherization robot (1) comprising:
a base (10),
a first drive module (2) for driving a first elongate flexible medical instrument (4) in translation along a longitudinal first direction, said first drive module:
being fixed relative to the base (10),
and comprising at least one pair of two drive surfaces (20) which are located face to face and which:
either move closer to each other in translation along a transverse second direction, in a first directional orientation, firstly so as to grip the first elongate flexible medical instrument (4) in order to then be able to drive it in translation along the longitudinal first direction,
or move away from each other in translation along the transverse second direction, in a second directional orientation that is opposite to the first directional orientation, in order to release the first elongate flexible medical instrument (4) so it is then free for extraction from the catherization robot (1),
a second drive module (3) for driving a second elongate flexible medical instrument (4) in translation along the longitudinal first direction, said second drive module being movable in translation along the longitudinal first direction relative to the first drive module (2), one end of the first elongate flexible medical instrument (4) being fixed to the second drive module (3),
wherein:
the maximum value of the separation distance between the two drive surfaces (20) of the first drive module (2), which is reached when the two drive surfaces (20) are moving away from each other, is:
greater than 20 mm,
or greater than 25 mm,
or greater than 30 mm,
or greater than 35 mm.

17. Catherization robot (1) comprising:
>a base (10),
a first elongate flexible medical instrument (4),
a first drive module (2) for driving in translation, along a longitudinal first direction, a first elongate flexible medical instrument (4) comprising a body (42) which is intended to enter the body of a patient and a manipulation area (41) which is intended to remain outside the body of the patient and which allows gripping this first elongate flexible medical instrument (4), said manipulation area (41) having a maximum transverse dimension that is greater than the maximum transverse dimension of the body (42) of the first elongate flexible medical instrument (4), the respective transverse dimensions of the manipulation area (41) and of the body (42) of the first medical instrument (4) being oriented in a transverse second direction which is orthogonal to the longitudinal first direction, said first drive module:
being fixed relative to the base (10),
and comprising at least one pair of two drive surfaces (20) which are located face to face and which:
either move closer to each other in translation along the transverse second direction, in a first directional orientation, firstly so as to grip the first elongate flexible medical instrument (4) in order to then be able to drive it in translation along the longitudinal first direction,
move away from each other in translation along the transverse second direction, in a second directional orientation that is opposite to the first directional orientation, in order to release the first elongate flexible medical instrument (4) so it is then free for extraction from the catherization robot (1), the separation distance between the two drive surfaces (20) then reaching a maximum value which is greater than said transverse dimension of said body (42) of the first elongate flexible medical instrument (4),
a second drive module (3) for driving a second elongate flexible medical instrument (4) in translation along the longitudinal first direction, said second drive module being movable in translation along the longitudinal first direction relative to the first drive module (2), one end of the first elongate flexible medical instrument (4) being fixed to the second drive module,
wherein:
said maximum value of the separation distance between the two drive surfaces (20) of the first drive module (2) is greater than said maximum transverse dimension of said manipulation area (41) of the first elongate flexible medical instrument (4).

18. Catherization robot (1) comprising:
a base (10),
a Y connector (5),
a first drive module (2) for driving a first elongate flexible medical instrument in translation along a longitudinal first direction, said first drive module:
being fixed relative to the base (10),
and comprising at least one pair of two drive surfaces (20) which are located face to face and which:
either move closer to each other in translation along the transverse second direction, in a first directional orientation, firstly so as to grip the first elongate flexible medical instrument (4) in order to then be able to drive it in translation along the longitudinal first direction,
or move away from each other in translation along the transverse second direction, in a second directional orientation that is opposite to the first directional orientation, in order to release the first elongate flexible medical instrument (4) so it is then free for extraction from the catherization robot (1),
a second drive module (3) for driving a second elongate flexible medical instrument (4) in translation along the longitudinal first direction, said second drive module:
being movable in translation along the longitudinal first direction relative to the first drive module (2), one end of the first elongate flexible medical instrument (4) being fixed to the second drive module,
and carrying the Y connector (5) within which the second elongate flexible medical instrument (4) can move in translation along the longitudinal first direction,
wherein:
the maximum value of the separation distance between the two drive surfaces (20) of the first drive module (2), which is reached when the two drive surfaces (20) are moving away from each other, is greater than the maximum transverse dimension of the Y connector (5) along the transverse second direction.

19. Catherization robot (1) according to claim 17, wherein:
said maximum value of the separation distance between the two drive surfaces (20) is:
greater than 1.25 times said maximum transverse dimension of said manipulation area (41) of the first elongate flexible medical instrument (4),
or greater than 1.5 times said maximum transverse dimension of said manipulation area (41) of the first elongate flexible medical instrument (4),
or greater than 1.25 times said maximum transverse dimension of the Y connector (5),
or greater than 1.5 times said maximum transverse dimension of the Y connector (5).

20. Catherization robot (1) according to claim 17, wherein:
said maximum value of the separation distance between the two drive surfaces (20) is:
less than 3 times said maximum transverse dimension of said manipulation area (41) of the first elongate flexible medical instrument (4),
or less than 2 times said maximum transverse dimension of said manipulation area (41) of the first elongate flexible medical instrument (4),
or less than 3 times said maximum transverse dimension of the Y connector (5),
or less than 2 times said maximum transverse dimension of the Y connector (5),
or less than 80 mm,
or less than 60 mm,
or less than 50 mm,
or less than 40 mm.

21. Catherization robot (1) according to claim 18, wherein:
said maximum value of the separation distance between the two drive surfaces (20) is:
greater than 1.25 times said maximum transverse dimension of said manipulation area (41) of the first elongate flexible medical instrument (4),
or greater than 1.5 times said maximum transverse dimension of said manipulation area (41) of the first elongate flexible medical instrument (4),
or greater than 1.25 times said maximum transverse dimension of the Y connector (5),
or greater than 1.5 times said maximum transverse dimension of the Y connector (5).

22. Catherization robot (1) according to claim 18, wherein:
said maximum value of the separation distance between the two drive surfaces (20) is:
less than 3 times said maximum transverse dimension of said manipulation area (41) of the first elongate flexible medical instrument (4),
or less than 2 times said maximum transverse dimension of said manipulation area (41) of the first elongate flexible medical instrument (4),
or less than 3 times said maximum transverse dimension of the Y connector (5),
or less than 2 times said maximum transverse dimension of the Y connector (5),
or less than 80 mm,
or less than 60 mm,
or less than 50 mm,
or less than 40 mm.

23. Catherization robot (1) according to claim 17, comprising:
an inlet opening in the body (42) of the first elongate flexible medical instrument (4), its transverse dimension in the transverse second direction being:
o greater than said maximum transverse dimension of said manipulation area (41) of the first elongate flexible medical instrument (4),
or greater than said maximum transverse dimension of the Y connector (5) if there is such,
an outlet opening in the body (42) of the first elongate flexible medical instrument (4), its transverse dimension in the transverse second direction being:
greater than said maximum transverse dimension of said manipulation body (42) of the first elongate flexible medical instrument (4)
or greater than said maximum transverse dimension of said manipulation area (41) of the first elongate flexible medical instrument (4)
or less than said maximum transverse dimension of the Y connector (5) if there is such.

24. Catherization robot (1) according to claim 16, wherein said manipulation area (41) of the first elongate flexible medical instrument (4) comprises one or more protuberances arranged in a manner that enables gripping the first elongate flexible medical instrument (4), said protuberances advantageously being fins.

25. Catherization robot (1) according to claim 16, wherein said two drive surfaces (20) are respectively carried by two rollers which rotate about two parallel axes but in directions opposite to each other and which are part of the first drive module (2).

26. Catherization robot (1) according to claim 16, wherein said two drive surfaces (20) are respectively carried by two endplates (20) which are movable in translation.

27. Catherization robot (1) according to claim 16, wherein:
said two drive surfaces (20) are respectively carried by two platforms (230):
which are movable in transverse translation along the transverse second direction,
which are part of the first drive module (2) which also comprises an actuator for driving said two platforms in translation along the transverse second direction (230).

28. Robot according to claim 27, wherein the actuator is formed by a worm screw system.

29. Catherization robot (1) according to claim 27, wherein the actuator is formed by an actuating cylinder.

30. Catherization robot (1) according to claim 16, wherein the first drive module (2) comprises at least two of said pairs of two drive surfaces (20), which are located one after the other along the longitudinal first direction.

31. Catherization robot (1) according to claim 17, wherein the first drive module (2) comprises at least two of said pairs of two drive surfaces (20), which are located one after the other along the longitudinal first direction.

32. Catherization robot (1) according to claim 16, wherein it is adapted to implement a method for moving an elongate flexible medical instrument (4) in a catherization robot (1), successively comprising:
a first step in which the body (42) of the first elongate flexible medical instrument (4) is driven in translation along the longitudinal first direction by at least one pair of drive surfaces (20),
a second step in which the body (42) of the first elongate flexible medical instrument (4) is driven in translation along the longitudinal first direction by a translational movement of the second drive module (3) relative to the first drive module (2) along the longitudinal first direction.

33. Catherization robot (1) according to claim 32, wherein the first step comprises:
a first sub-step in which the body (42) of the first elongate flexible medical instrument (4) is driven in translation along the longitudinal first direction by at least two of said pairs of drive surfaces (20),
a second sub-step in which the body (42) of the elongate flexible medical instrument (4) is driven in translation along the longitudinal first direction by only one of said pairs of drive surfaces (20).

34. Catherization robot (1) according to claim 17, wherein it is adapted to implement a method for moving an elongate flexible medical instrument (4) in a catherization robot (1), successively comprising:
a first step in which the body (42) of the first elongate flexible medical instrument (4) is driven in translation along the longitudinal first direction by at least one pair of drive surfaces (20),
a second step in which the body (42) of the first elongate flexible medical instrument (4) is driven in translation along the longitudinal first direction by a translational movement of the second drive module (3) relative to the first drive module (2) along the longitudinal first direction.

35. Catherization robot (1) according to claim 34, wherein the first step comprises:
a first sub-step in which the body (42) of the first elongate flexible medical instrument (4) is driven in translation along the longitudinal first direction by at least two of said pairs of drive surfaces (20),
a second sub-step in which the body (42) of the elongate flexible medical instrument (4) is driven in translation along the longitudinal first direction by only one of said pairs of drive surfaces (20).
